SEQUENCE LISTING (9 sequences)

SEQ ID NO: 1 - protein, 17 aa, Artificial Sequence
  Description of Artificial Sequence Synthetic peptide
KMIHQHLSSRTHGSEDS

SEQ ID NO: 2 - protein, 15 aa, Artificial Sequence
  Description of Artificial Sequence Synthetic peptide
NVSIKKLKRKPPSTN

SEQ ID NO: 3 - protein, 15 aa, Artificial Sequence
  Description of Artificial Sequence Synthetic peptide
LGTLVHKSSSQGQDR

SEQ ID NO: 4 - protein, 15 aa, Artificial Sequence
  Description of Artificial Sequence Synthetic peptide
TNAGRRQKHRLTCPS

SEQ ID NO: 5 - protein, 15 aa, Artificial Sequence
  Description of Artificial Sequence Synthetic peptide
CDSYEKKPPKEFLER

SEQ ID NO: 6 - protein, 16 aa, Artificial Sequence
  Description of Artificial Sequence Synthetic peptide
CFQKAQLKSANTGNNE

SEQ ID NO: 7 - DNA, 21 bp, Artificial Sequence
  Description of Artificial Sequence Synthetic oligonucleotide
agttgaggggagtttcccagg

SEQ ID NO: 8 - DNA, 22 bp, Artificial Sequence
  Description of Artificial Sequence Synthetic oligonucleotide
cggaccctttcaggggagttga

SEQ ID NO: 9 - DNA, 67 bp, Artificial Sequence
  Description of Artificial Sequence Synthetic oligonucleotide
acaacgcgtgagctctctagaaagcatcatctcaacactaacttgataattaagtgcctcgagcaca

(19) United States
(12) Patent Application Publication
(10) Pub. No.: US 2009/0214549 A1
(43) Pub. Date: Aug. 27, 2009

(54) Antigenic Epitopes Of Inteleukin-21, Related Antibodies And Their Use In Medical Field

(76) Inventor: Giovanni Monteleone, Rome (IT)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/791,764

(22) PCT Filed: Nov. 24, 2005
(86) PCT No.: PCT/IT2005/000691

§ 371 Date: Apr. 25, 2008

(30) Foreign Application Priority Data
Nov. 29, 2004 (IT) RM2004A000586

(51) Int. Cl. A61K 39/395; C07K 16/24; G01N 33/566; A61P 17/06; A61P 29/00; A61P 37/00; A61P 1/00; C12N 5/00

(52) U.S. Cl. 424/139.1; 530/387.9; 436/501; 435/375

(57) ABSTRACT

The present invention relates to antigenic epitopes of interleukin-21, related antibodies and their use in medical field, in particular for the treatment of immune-inflammatory diseases characterized by an enhanced production and/or activity of IL-21, such as for example chronic inflammatory bowel diseases (IBD), celiac disease and psoriasis.

The present invention relates to antigenic epitopes of interleukin-21 (IL-21) and related neutralizing IL-21 antibodies, and their use for the treatment of patients with immune-inflammatory diseases characterized by increased production or activity of IL-21, such as Crohn's disease (CD), ulcerative colitis (UC), celiac disease and psoriasis.

During the last decade, several studies carried out in humans and animal models have advanced our understanding of pathogenesis of immunomediated diseases, and contributed to optimize the therapeutic intervention in patients suffering from these pathologies. Thus, it has been shown that T lymphocytes play a major pathogenic role in disease, such as psoriasis, rheumatoid arthritis, bronchial asthma, primitive biliary cirrhosis, CD, UC, celiac disease, Helicobacter pylori (Hp)-associated gastric disease, multiple sclerosis (Nickoloff 2004, Firestein 2004; Sollid 2002; Podolsky 2002). A careful molecular analysis of the events underlying the inflammatory process has allowed to verify that the pathogenic action of T lymphocytes is mostly related to their ability to synthesize several inflammatory molecules, among which cytokines (or interleukins) A relevant example is represented by the production of tumor necrosis factor-alfa (TNF-α), a cytokine that is able to accomplish multiple inflammatory effects by acting on monocytes, dendritic cells, fibroblasts, myofibroblasts, endothelial and epithelial cells (Campbell 2003).

A better knowledge of the natural history of these inflammatory diseases, and of their pathophysiological mechanisms, has therefore contributed to improve the therapeutic choices, although the pharmacologic intervention for each of these pathologies is merely symptomatic and not curative. In most patients, the resolution of the inflammatory activity and the induction of the clinical remission require a treatment with corticosteroids. However, it is widely known that such drugs are effective in nearly 50% of patients, and that half of these patients require a continuous treatment for maintaining clinical remission. Unfortunately, the beneficial effects of steroids come at the expense of frequent and often severe adverse effects (Spahn 1997, Scholmerich 2004). Even the immunosuppressive treatment, which often accompanies or replaces corticosteroids, is not always sufficient to limit the inflammation and to control the symptoms. Moreover, it has the disadvantage of distinct contraindications and severe adverse effects (Podolsky, 2002).

The new drug generation that became available in the 1990's, are biological agents. These are biotherapies aimed at controlling specific inflammatory “pathways”, through the use of recombinant human proteins, monoclonal chimeric humanized antibodies and fusion proteins. In this context, a compound which exhibits a good efficacy in inhibiting immunoinflammatory processes is the monoclonal anti-TNF-α antibody (Seegers et al., 2002). This drug is able to contain the inflammation in about 50-70% of patients. Nevertheless, the incidence of averse effects, such as reactivation of latent microbial infections and hypersensitivity phenomena, has been reported to increase with repeated treatments. The latter phenomenon could rely on the fact that the antibody blocks a cytokine which has multiple biological functions. In fact, beyond its inflammatory effect, TNF-α plays a role in mechanisms involved in the induction and maintenance of immunological tolerance. It is thus plausible that blocking TNF-α could paradoxically encourage excessive immunological reactions. Additionally, more than one third of treated patients produce antibodies against the compound that seem to reduce the effectiveness of the drug (Sandborn, 2002). Overall these observations suggest the necessity of additional studies aimed at identifying new compounds for the management of patients with the above mentioned pathologies (Fiocchi, 2001).

Inteleukin-21 (IL-21) is a cytokine of recent identification, mainly produced by activated CD4+ T lymphocytes (Habib 2003, Parrish-Novak 2002). Biological effects of IL-21 are mediated through a membrane receptor, named IL-21R, which shows homology with αsubunit of IL-2 and IL-15 receptors, and interacts with the receptor subunit γ-chain (Collins 2003, Zhang 2003). Up to now, such a receptor has been shown on T and B cells, natural killer lymphocytes, monocytes and dendritic cells.

Analysis of the signal transduction cascade induced by interaction of IL-21 with its receptor has shown that this cytokine is able to trigger some intracellular signals and to allow specific immune-inflammatory responses, strictly dependent on cell type analysed. Particularly, several studies carried out in B cells have shown that IL-21 can either promote or inhibit cell death programs in human or murine cells respectively (Mehta 2003, Jin 2004). Similarly, it has been shown that human peripheral blood T lymphocytes produce high levels of interferon-gamma (IFN-γ), indicative of a Th1 response, following IL-21 stimulation, whereas in some murine models IL-21 seems to be expressed at higher level in Th2 lymphocytes and promote the differentiation of this cell type (Ma 2003, Wurstel 2002). Experiments carried out in animal models of carcinogenesis and diabetes have either confirmed the ability of IL-21 to modulate T lymphocytes/natural killer cell activities and cytokine production, and have underlined the potential role of this molecule in the development and perpetuation of immune-inflammatory process (Gallegos 2004). It is thus conceivable that the modulation of IL-21 biological effects could represent a new and promising therapeutic approach for all the immune-inflammatory diseases characterized by an altered IL-21 production and/or activity.

As above mentioned, during CD, a chronic inflammatory disease mainly localized at distal ileum and right colon, intestinal mucosa is heavily infiltrated with T cells (Podolsky 2002). There is also evidence that T cells are differentiated at the mucosal level into Th1 lymphocytes, and produce high levels of INF-γ and TNF-α. Th1 cell differentiation seems to be dependent on locally-produced cytokines, such as IL-12 (Neurath M F 1996, Monteleone 1997). Studies carried out both in humans and animal models of IBD have also shown that activation of Th1 lymphocytes by IL-12 triggers a cascade of molecular events that lead to tissue damage (Neurath MF 1996, Monteleone 1999). Essential mediators of the damage are matrix metalloproteinases (MMPs) (Monteleone, 1999), a family of proteases that can degrade several component of the extracellular matrix, thus causing mucosal degradation and disruption of epithelial structure. Consistently, a marked increase of some MMPs, including MMP-1 (interstitial collagenase), MMP-2 (gelatinase A), MMP-3 (stromelysin) and MMP-9 (gelatinase B), has been documented in gastric and intestinal ulcers, as well as in inflamed mucosal areas of patients with Hp infection, CD and UC (Heuschkel 2000, Saarialho-Kere 1996). In addition, neutralization of MMPs has been shown to be sufficient to limit the mucosal degradation triggered by IL-21-activated Th1 cells in explants of foetal gut (Monteleone, 1999).

The importance of MMP's in the pathogenesis of immune-inflammatory diseases is not limited to CD and UC, since there is evidence of their involvement also in psoriasis, rheumatoid arthritis, diabetes, multiple sclerosis (Kane 2004, Wall 2003).

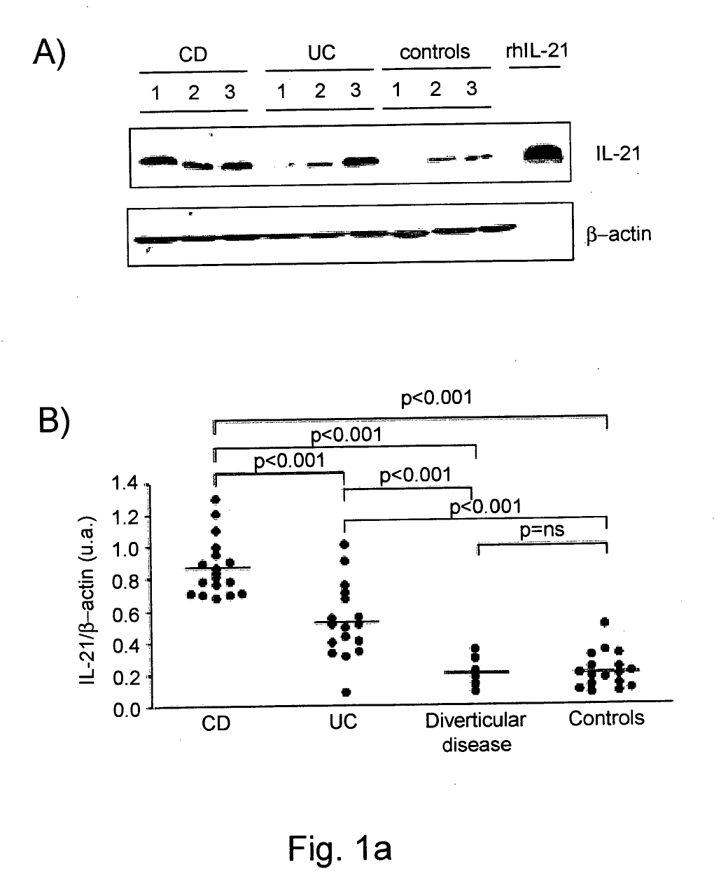

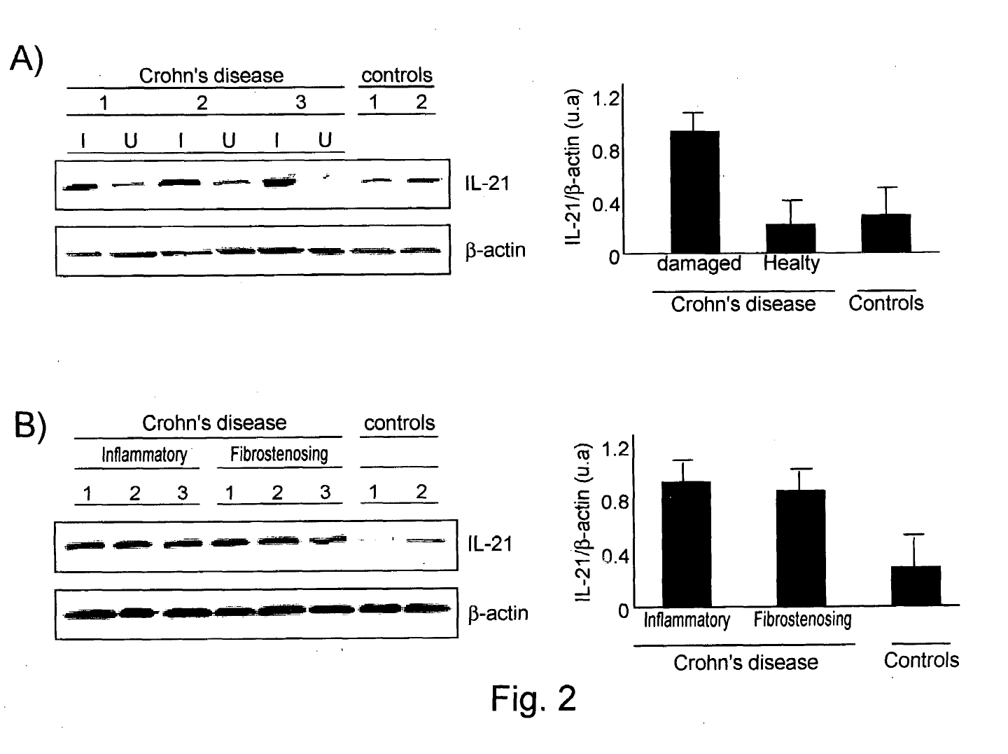

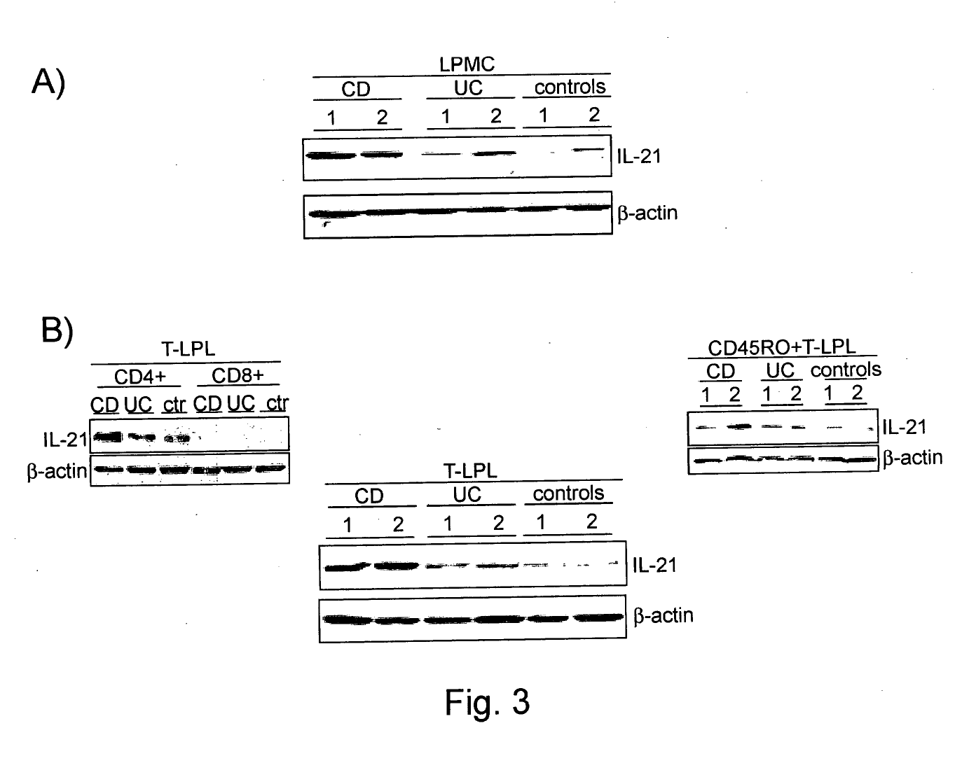

The author of the present invention shows now that in the intestinal mucosa of patients with CD there is a sustained IL-21 production (FIG. 1), and such an increase is restricted to the active lesions areas, and does not depend on the lesion type (FIG. 2). Enhanced IL-21 expression in CD does not simply reflect the increased infiltration of the mucosa with T lymphocytes. In fact, an enhanced IL-21 synthesis is documented also in protein extracts of CD4+ T cells and CD45RO+ lymphocytes from CD patients in comparison to controls (FIG. 3).

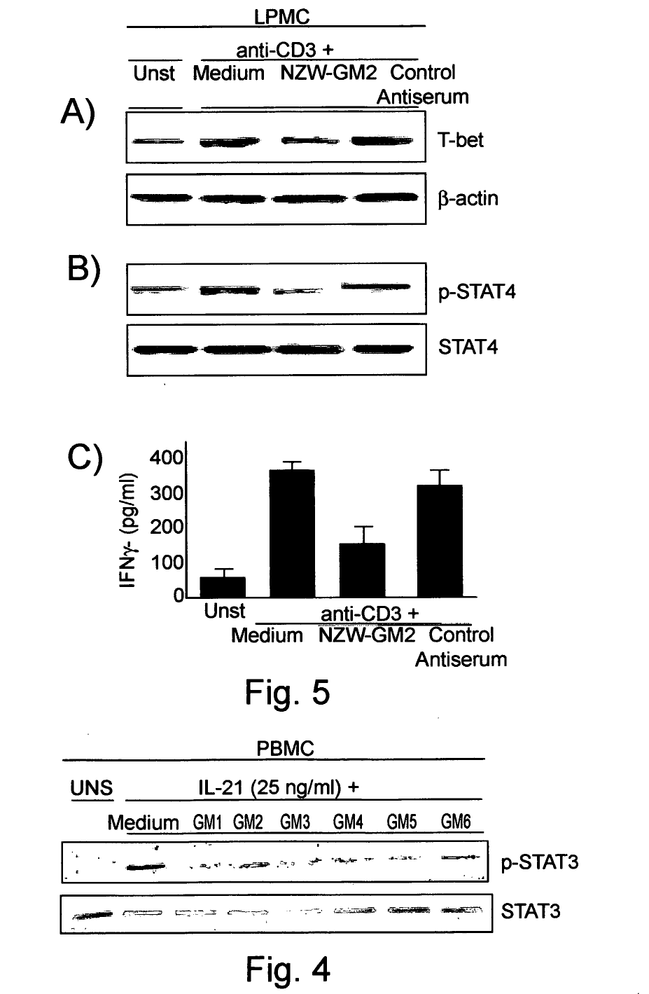

There is no doubt that Th1 cells polarization and stabilization is strictly dependent on the activity of molecules enhancing IL-12 activity. To examine whether IL-21 could be involved in the modulation of this response, the author of the present invention has designed, developed and tested the effectiveness of 6 different antibody molecules (antisera) directed against IL-21. To produce these antibodies NZW rabbits immunization with specific human IL-21 sequences was employed. The IL-21 activity inhibition of each antiserum has been depicted in FIG. 4. Particularly, each antiserum effect on the IL-21-mediated induction of Stat3 phosphorylation (p-Stat3) in human peripheral blood mononuclear cells (PBMC) is shown. The neutralizing effect of each antiserum was also confirmed using Jurkat cells, a T cell line (not shown). Moreover, It has been shown that the neutralization of IL-21 activity in intestinal lamina propria mononuclear cells (LPMC) cultures from CD patients, through the use of GM2 antiserum (which during PBMC and Jurkat experiments has consistently and equally inhibited IL-21 activity), is associated with a marked reduction in the expression of transcription factors essential for Th1 cells differentiation, such as Stat4 and T-bet, and inhibition of IFN-γ synthesis (FIG. 5).

The marked accumulation of T cells in the inflamed mucosa of CD patients is believed to rely partly on the resistance of such cells against apoptotic stimuli. There is also evidence that such a phenomenon plays a key role in maintaining the mucosal inflammatory state, since drugs that are able to increase T cell susceptibility to apoptosis stimuli, such as the anti-TNF-α, promote the resolution of the ongoing tissue inflammatory process. The exact mechanism underlying the resistance of T cells against apoptosis during IBD is not yet known, even if locally released cytokines seem to be involved.

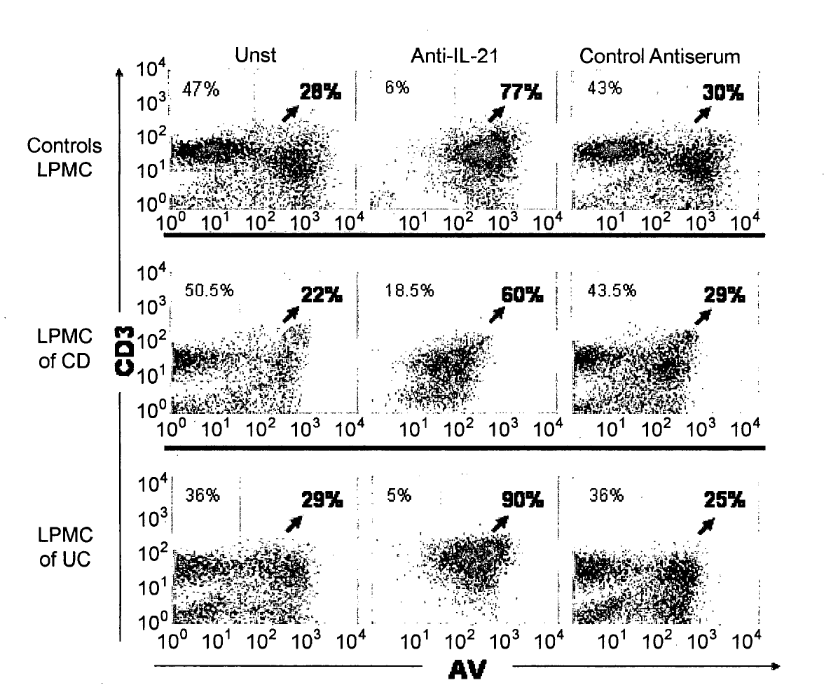

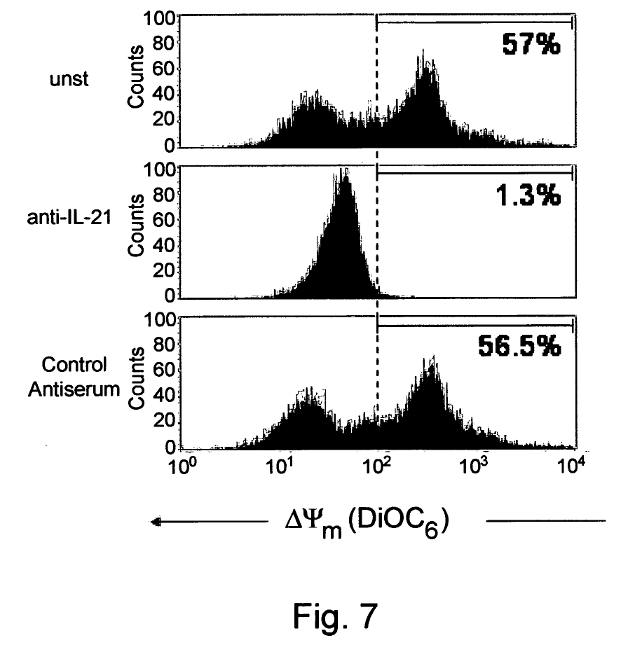

The author of the present invention has shown that blocking IL-21 activity, by using the antisera that are object of the present invention, associates with a marked and significant reduction of CD3+ T cell survival in cultures of LPMC isolated from both CD and UC patients (FIG. 6). Similar data were obtained when cultures o intestinal lamina propria CD3+ T cells were used. Indeed, fluorimetric analysis of CD3+ T cells showed that the anti-IL-21 antiserum GM2 caused a significant increase in the percentage of annexin V (AV) (28,5±8) or propidium iodide (IP) (7±1,4) or both (41±7,6) in comparison to untreated cells (AV: 11±1; IP: 1,5±0,16; AV-IP:20,8±5,7) or cells treated with a control serum (AV: 11,6±0,69; IP: 2±0,7; AV-IP:21±5,77) (P<0.01). A similar effect was also observed when the anti-IL-21 was added to CD4+ T cell culture. Such effect was associated with a loss of mitochondrial membrane potential (FIG. 7).

As above stated the enhanced expression of IL-21 has been shown also in the inflamed areas of patients with UC (FIG. 1), a chronic inflammatory disease characterized by a marked mucosal infiltration of T cells not polarized toward the Th1 subtype. Therefore, this observation suggests the possibility that IL-21 could regulate multiple inflammatory pathways other than controlling Th1 polarization.

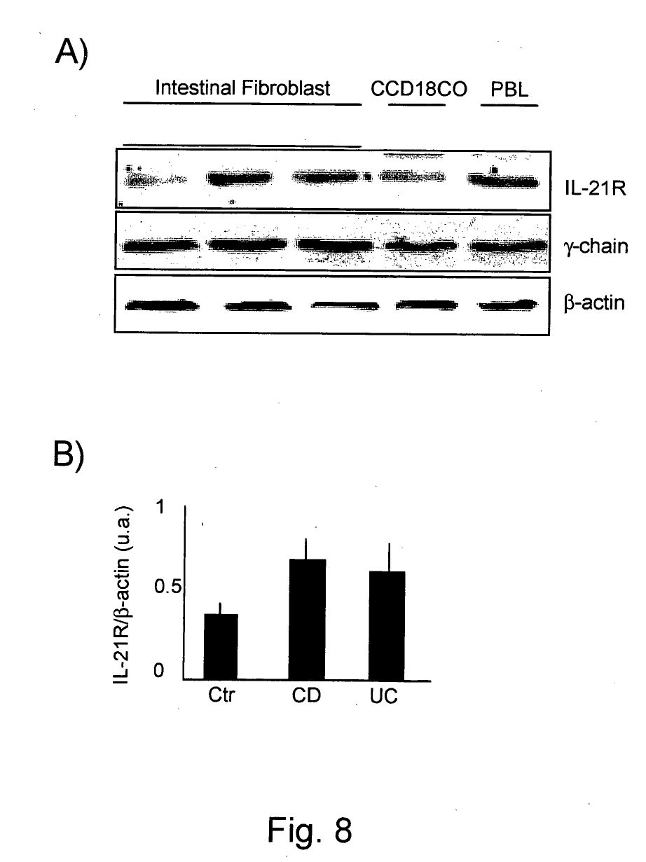

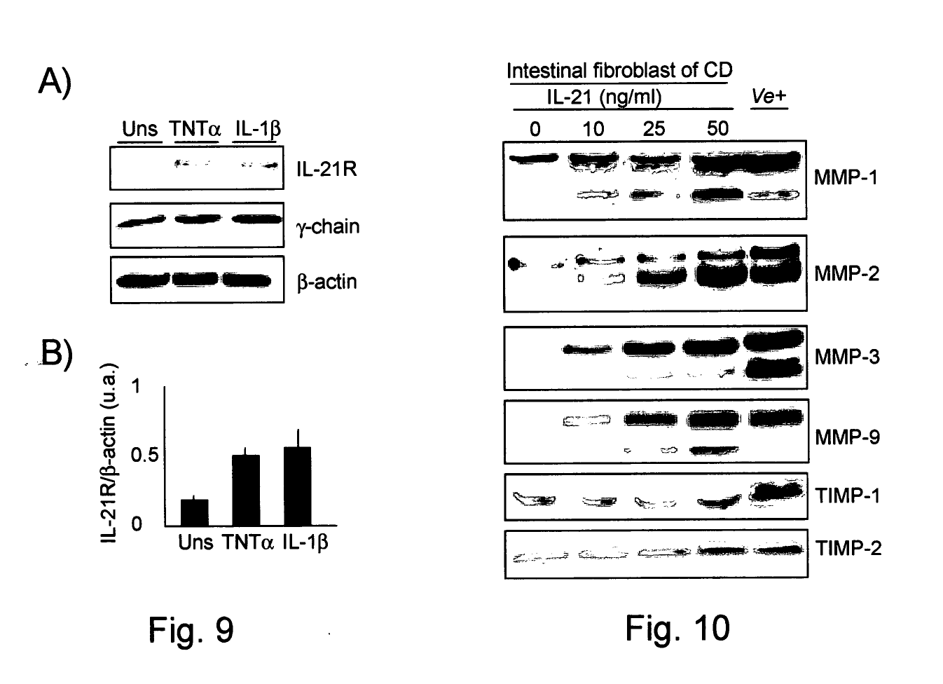

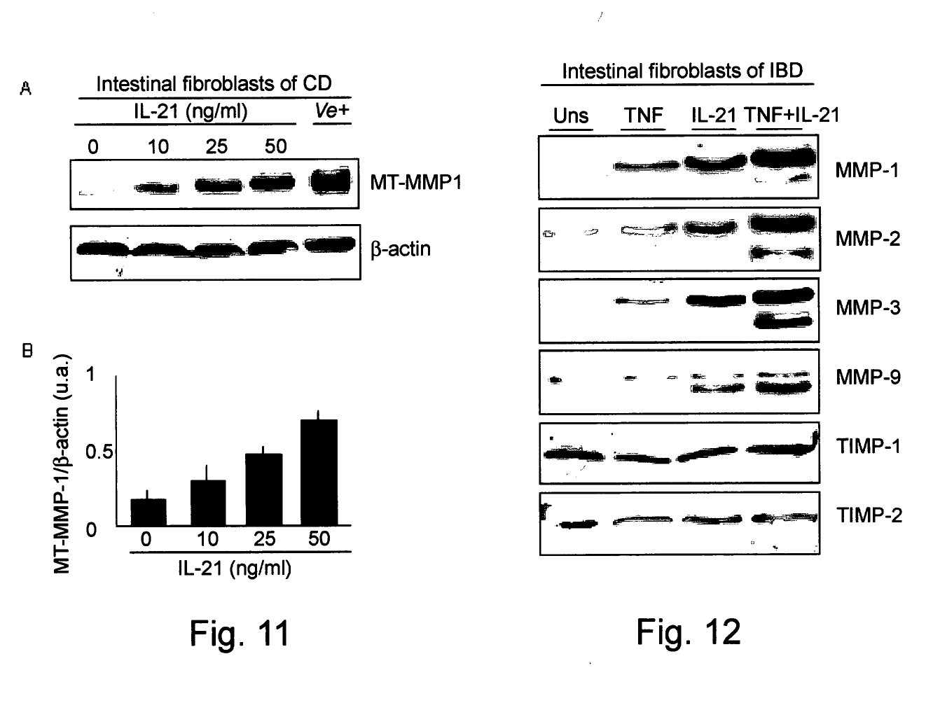

To ascertain new IL-21 biological functions, first it has been characterised the expression of IL-21 receptor (IL-21R) in human gut mucosal cells. Data from these experiments indicate that IL-21R expression is not restricted to immune cells, but surprisingly IL-21R is also expressed by non-immune cells, such as intestinal myo-fibroblasts (FIG. 8). Such an expression can be enhanced by inflammatory stimuli, such as TNF-α or IL-1β(FIG. 9). Second, it has been ascertained that such cells are responsive to IL-21 stimulation. In particular, the addition of IL-21 to cultures of intestinal myo-fibroblasts isolated from both healthy subjects (normal controls) and IBD patients resulted in a massive secretion of MMP-1, MMP-2, MMP-3 and MMP-9 (FIG. 10), without affecting the secretion of tissue inhibitors of MMPs, like TIMP-1 and TIMP-2. The effect of IL-21 on MMP production was seen also in fetal gut fibroblasts lines (CCD18CO) (data not shown) and was dose-dependent (FIG. 10). Furthermore, it has been shown that IL-21 enhances the expression of membrane MMP, such as MT-MMP-1 (FIG. 11), whose activity is essential for MMP-2 activation. Finally, the effect of IL-21 on MMPs synthesis was enhanced by concomitant stimulation with TNF-α (FIG. 12), thus suggesting that these two cytokines cooperate in the modulation of MMPs production.

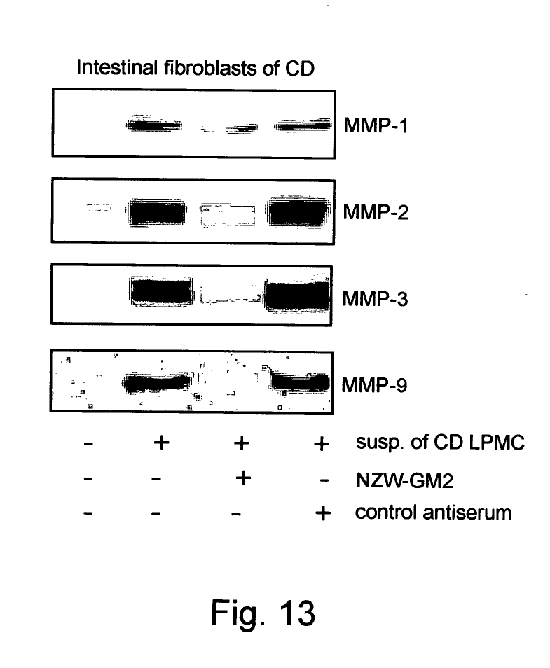

To evaluate whether CD LPMC-derived IL-21 can modulate MMPs production, intestinal fibroblasts isolated from CD mucosal specimens were cultured in the presence or absence of CD LPMC supernatants (containing IL-21, FIG. 3) with or without the addition of an anti-IL-21 or a control antiserum for 48 hours, and MMPs production was assessed by Western blotting. As shown in FIG. 13, the addition of CD LPMC supernatants to the myo-fibroblast cultures resulted in enhanced synthesis of all MMPs, and such effect was inhibited by anti-IL-21. This demonstrates that IL-21 produced by CD LPMC is biologically active and is able to enhance MMPs synthesis.

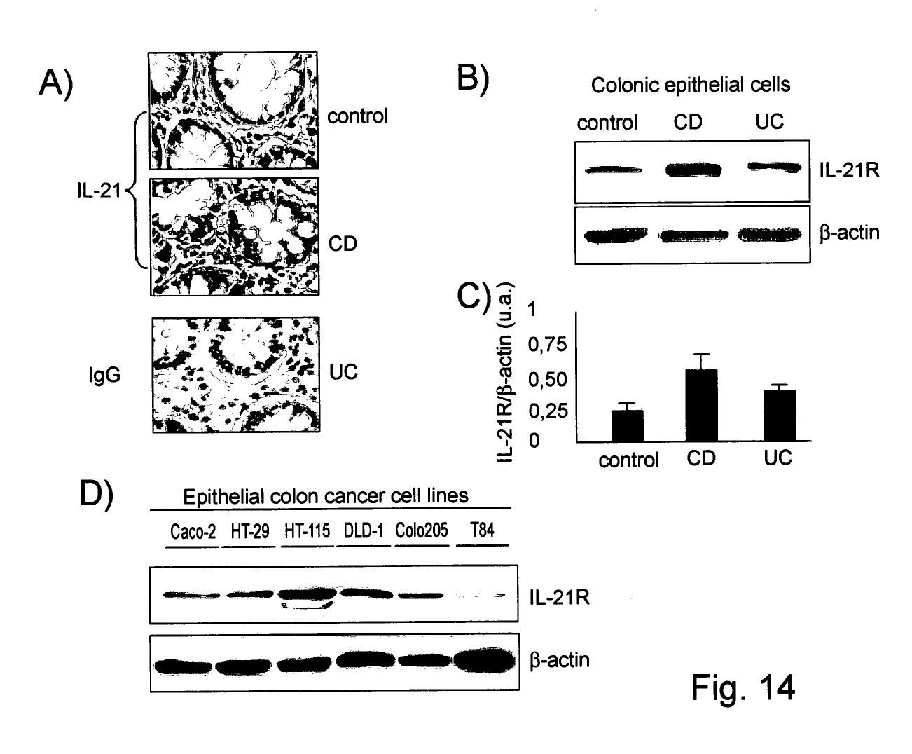

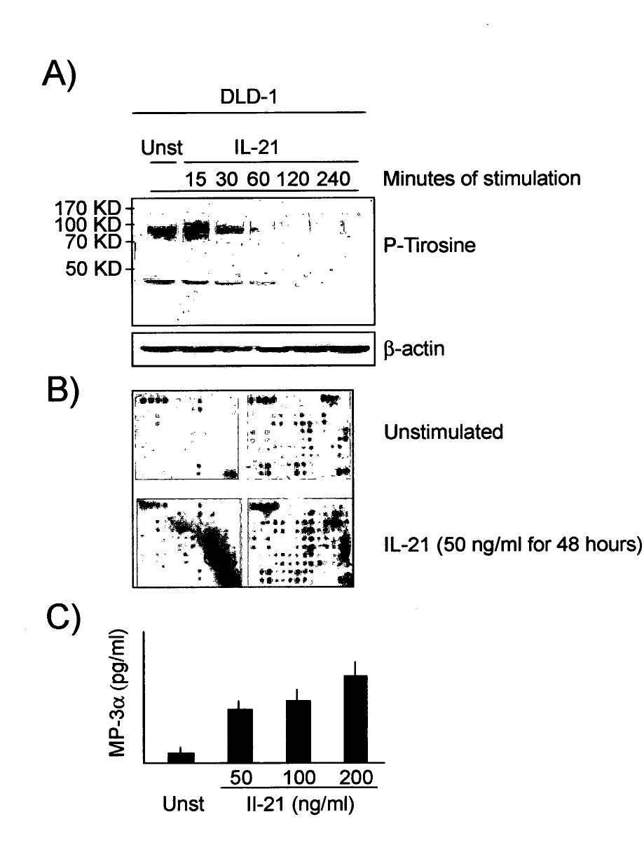

More surprisingly it has been shown that IL-21R is expressed by gut epithelial cells, and that such expression is enhanced during IBD, especially in CD (FIG. 14). According to these data, a constitutive IL-21R expression was documented in several colon cancer epithelial cell lines (FIG. 14). It has been further shown that gut epithelial cells are responsive to IL-21. In fact, stimulation of epithelial cell lines with IL-21 was followed by a marked change in the content of tyrosine phosphorylated intracellular proteins (FIG. 15, panel A). To test whether IL-21 changes also the expression of pro- or anti-inflammatory molecules, culture supernatants of the same epithelial cell lines, either left unstimulated or stimulated with IL-21, were assayed for the expression of 120 different proteins. The stimulation with IL-21 was associated with an enhanced secretion of MIP-3α (FIG. 15, panel B), a protein produced in excess from epithelium during CD and UC, and other immune-inflammatory diseases such as psoriasis, and involved in the recruitment of lymphocytes and dendritic cells to the inflamed tissues (Kwon 2002, Dieu-Nosjean 2000). These data were then confirmed by ELISA (FIG. 15, panel C).

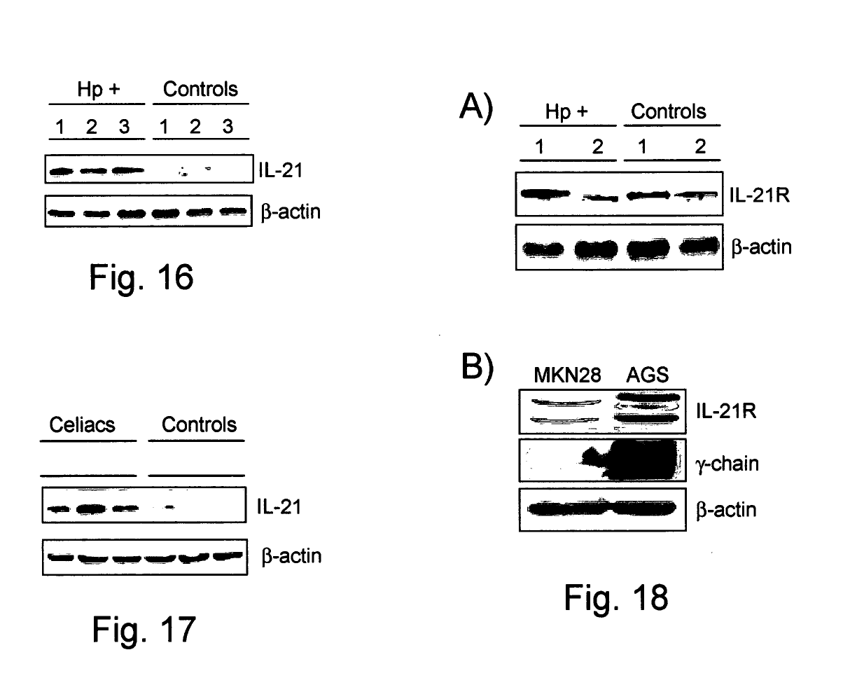

To confirm the potential role of IL-21 in the pathogenesis of other gastrointestinal immune-inflammatory diseases, the author of the present invention has also demonstrated that IL-21 is over-expressed in the stomach of patients with gastritis correlated with Hp infection and in the gut of celiac disease patients, two pathologies characterized by a predominant mucosal Th1 cell response (FIGS. 16 and 17).

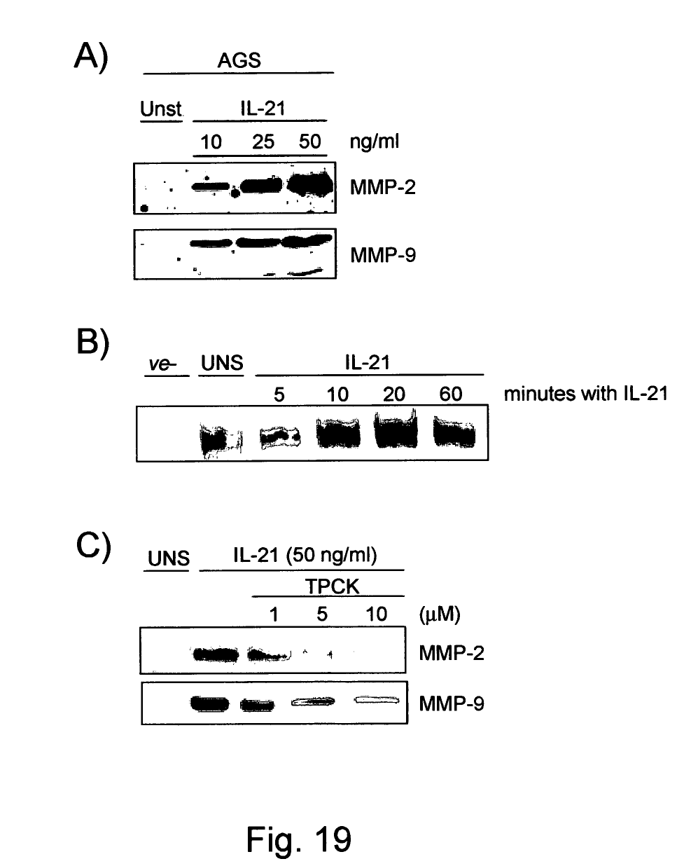

IL-21R expression was documented also in gastric epithelial cells, isolated from patients with or without Hp infection, as well as in gastric cancer epithelial cells lines (AGS and MKN) (FIG. 18). Consistently, stimulation of AGS with IL-21 was associated with enhanced synthesis of MMP-2 and MMP-9, two gelatinases produced in excess in the stomach of patients with Hp infection (FIG. 19, panel A) (Mori, 2004).

Analysis of molecular mechanisms underlying the IL-21 effect on MMP2 and MMP9 production revealed that IL-21 activated NF-kB in AGS cells (FIG. 19, panel B) and that NF-kB inhibition through the use of a commercially available product (TPCK) was associated with a marked inhibition of the IL-21-mediated MMP-2 and MMP-9 synthesis (FIG. 19, panel C).

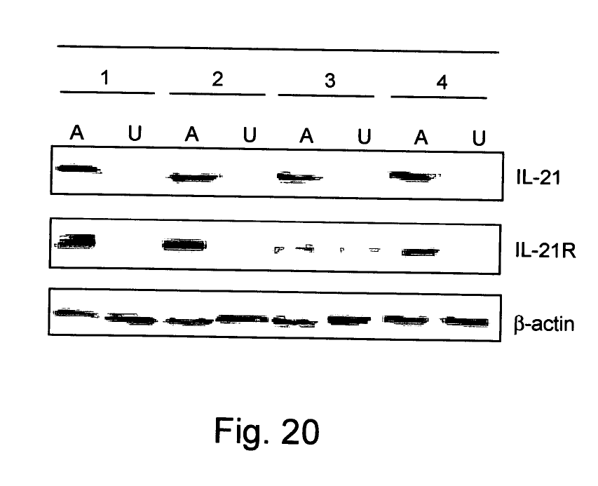

Finally, it has been shown that IL-21 and IL-21R are strongly induced in affected areas of patients with psoriasis (FIG. 20).

Overall these data show that neutralization of IL-21 activity could be a rational objective for inhibiting the ongoing inflammation in pathologies associated with a dysregulated synthesis/activity of this cytokine.

It is therefore an object of the present invention antigenic epitopes belonging to the human IL-21 sequence comprising the following sequences:

1. KMI HQH LSS RTH GSE DS (SEQ ID NO:1), comprising amino acids 146-162 of human IL-21, and named GM1;
2. NVS IKK LKR KPP STN (SEQ ID NO:2), comprising amino acids 97-111 of human IL-21, and named GM2;
3. LGT LVH KSS SQG QDR (SEQ ID NO:3), comprising amino acids 20-34 of human IL-21, and named GM3;
4. TNA GRR QKH RLT CPS (SEQ ID NO:4), comprising amino acids 110-124 of human IL-21, and named GM4;
5. CDS YEK KPP KEF LER (SEQ ID NO:5), comprising amino acids 125-139 of human IL-21, and named GM5;
6. CFQ KAQ LKS ANT GNN E (SEQ ID NO:6), comprising amino acids 78-93 of human IL-21, and named GM6; or portions of at least 5 amino acids thereof.

Another object of the present invention is represented by oligonucleotidic sequences encoding for the antigenic epitopes as above defined.

Furthermore, the invention relates to the use of epitopes as above defined for the preparation of anti-IL-21 antibodies. Particularly, antibodies could be polyclonal or monoclonal, antibody fragments, chimeric or single chain antibodies.

Further objects of the present invention are specific and selective antibodies for at least one of the antigenic epitopes according to the invention for human IL-21 neutralization, directed against one of the following peptide sequences of the human IL-21 protein:

1. KMI HQH LSS RTH GSE DS (SEQ ID NO:1), comprising amino acids 146-162 of human IL-21, and named GM1;
2. NVS IKK LKR KPP STN (SEQ ID NO:2), comprising amino acids 97-111 of human IL-21, and named GM2;
3. LGT LVH KSS SQG QDR (SEQ ID NO:3), comprising amino acids 20-34 of human IL-21, and named GM3;
4. TNA GRR QKH RLT CPS (SEQ ID NO:4), comprising amino acids 110-124 of human IL-21, and named GM4;
5. CDS YEK KPP KEF LER (SEQ ID NO:5), comprising amino acids 125-139 of human IL-21, and named GM5;
6. CFQ KAQ LKS ANT GNN E (SEQ ID NO:6), comprising amino acids 78-93 of human IL-21, and named GM6.

These antibodies may be polyclonal (Ab) or monoclonal (mab), or antibody fragment (Fab Fab′, Fab(ab′)2) or chimeric, humanized, or single chain (scFv) antibodies. Antibodies may be mammals antibody and preferably are of human, murine, rabbit, goat origin. The necessary variations to enhance the stability of the molecule, to prevent its degradation, or to reduce the risk of side effects are also comprised within the scope of the present invention. Antibodies according to the present invention may be labelled with a fluorescent dye, a radioisotope or a drug. Antibodies may be also employed as reactant in experimental fields, for example in IL-21 expression neutralization or characterization assays.

Antibodies according to the present invention may be advantageously used in medical field. The invention further concerns the use of the antibodies according to the invention for the preparation of a medicament for the treatment or in vitro diagnosis of immune-inflammatory diseases associated with an altered IL-21 expression such as, for example, chronic inflammatory bowel diseases (IBD), celiac disease, psoriasis, or autoimmune thyroiditis. Particularly, chronic inflammatory bowel diseases may be Crohn's disease (CD) or ulcerative colitis (UC).

It is another object of the present invention, a pharmaceutical composition comprising at least one of the antibodies as above defined as active principle together with one or more pharmaceutically acceptable co-adjuvants and/or excipients, that are well known to people skilled in the art. Preferably, the composition is administered by topic, systemic or oral route.

Furthermore, the present invention relates to a diagnostic kit comprising at least one of the antibodies and/or epitopes as above defined according to the invention. Preferably, the diagnostic kit according to the invention employs techniques such as ELISA, Western blotting, immunohistochemistry or cytofluorimetry for the diagnosis of immune-inflammatory diseases associated with an altered IL-21 expression such as, for example, chronic inflammatory bowel diseases (IBD), like CD and UC, celiac disease, psoriasis, or autoimmune thyroiditis. Such diagnostic kits are useful for the neutralization or characterization of the in vitro or in vivo IL-21 expression.

The present invention is now described, for illustrative but not limitative purposes, according to its preferred embodiments, with particular reference to the figures of the enclosed drawings, wherein:

FIG. 1, panel A, shows the expression of IL-21 (upper blot) and β-actin (lower blot) in total protein extracts from intestinal mucosal samples from 3 patients with CD, 3 with UC and 3 normal controls, as measured by Western blotting. The lower panel shows the IL-21/β-actin protein ratio in each subject (represented by each point in the graph), expressed in arbitrary densitometric units. Horizontal bar indicates the median of the values for each group;

FIG. 2, panel A, shows the analysis of the expression of IL-21 (upper blot) and β-actin (lower blot) in total protein extracts from intestinal mucosal samples from 3 patients with CD, and 2 normal controls, evaluated by Western blotting. For each patient with CD, samples were taken from both inflamed (affected) and uninflamed areas. The right panel shows the IL-21/β-actin protein content ratio, measured in different patients and healthy controls and expressed as mean±standard deviation of arbitrary densitometry units. Panel B of FIG. 2 shows the IL-21 protein content (upper blot) and β-actin (lower blot) in total protein extracts from intestinal mucosal samples from 3 patients with inflammatory CD, 3 patients with fibrostenosing CD, and 2 normal controls. The right panel shows the IL-21/β-actin protein ratio, evaluated in different patients and healthy controls and expressed as mean±standard deviation of arbitrary densitometric units;

FIG. 3, panel A, shows the expression of IL-21 (upper blot) and β-actin (lower blot) in total protein extracts in intestinal lamina propria mononuclear cells (LPMC) from 2 patients with CD, 2 with UC and 2 normal controls, as measured by Western blotting. Panel B shows a representative Western blot of IL-21 and β-actin in total protein extracts isolated from intestinal lamina propria (LPL) T cells of 2 patients with CD, 2 with UC and 2 normal controls. Left inset shows IL-21 expression in CD4+ o CD8+ LPLs isolated from 1 patient with CD, 1 patient with UC and one normal control. The right inset shows the IL-21 protein content in CD45RO+ LPL purified from the intestine of 2 patients with CD, 2 with UC and 2 normal controls;

FIG. 4 shows the inhibitory effect of each anti-IL-21 antiserum (sequences GM1, GM2, GM3, GM4, GM5, GM6) on the induction of phosphorylation of the transcription factor Stat3 (pStat3) in human peripheral blood mononuclear cells (PBMC) stimulated with human recombinant IL-21. The lower blot shows total Stat 3 content in the same samples. Both pStat3 and total Stat3 were evaluated by Western blotting;

FIG. 5 shows the inhibitory effect of the GM2 antiserum on the expression of the transcriptional factors associated with Th1 response (T-bet and Stat4) and interferon-gamma synthesis (IFN-γ) in LPMC isolated from the intestine of CD patients. T-bet (A) and phosphorylated and total Stat4 (B) contents were analysed by Western blotting of total protein extracts, while IFN-γ secretion (C) in the culture supernatants was evaluated by ELISA, using a commercial kit;

FIG. 6 shows the percentage of CD3+ cells and/or annexin V (AV), as measured by cytofluorimetry in LPMC samples isolated from the gut of normal subjects, and patients with CD and UC, and cultured in presence or absence of anti-IL-21 antiserum (GM2) or control serum for 20 hours;

FIG. 7 shows the DIOC6 content as measured by cytofluorimetry in CD4+ T cells isolated from the intestine of a CD patient and cultured in presence or absence of anti-IL-21 antiserum (GM2) or control serum for 4 hours;

FIG. 8 shows a representative Western blotting for IL-21R and γ-chain receptor subunit in total protein extracts from myofibroblasts isolated from the colon of one normal subject (control), one patient with CD and one with UC, from fetal gut fibroblast cell line (CCD18CO), and from peripheral blood lymphocytes (PBL) of a CD patient. Panel B of the Figure shows the IL-21R/β-actin content ratio in protein extracts prepared from intestinal myofibroblasts from 8 patients with CD, 8 with UC and 8 normal controls. Values are expressed in arbitrary densitometric units and indicate mean±standard deviation of all experiments;

FIG. 9 shows a representative Western blotting for IL-21R in total protein extract from primary myofibroblasts isolated from the colon of one normal subject and cultured with medium alone (unst) or stimulated with TNF-α (20 ng/ml) or IL-1β (20 ng/ml) for 24 hours. Panel B of the figure shows the IL-21R/β-actin content ratio in protein extracts prepared from stimulated intestinal myofibroblasts as above indicated. Values are expressed in arbitrary densitometric units and indicate mean±standard deviation of 3 separate experiments;

FIG. 10 shows the IL-21 stimulatory effect on the synthesis of MMP (MMP-1,2,3 and 9) in intestinal myofibroblasts isolated from a CD patient. The IL-21 effect on MMPs is dose-dependent and is not associated with any change in the production of tissue inhibitors of MMPs (TIMP1 and TIMP2). Similar results were obtained with myofibroblasts isolated from the intestine of UC patients. Ve+=protein extracts from the colon of a patient with CD were used as positive control;

FIG. 11 shows the effect of IL-21 on the synthesis of MT-MMP1 in protein extracts prepared from intestinal myofibroblasts isolated from a CD patient and cultured with the medium alone or with graded doses of IL-21. The IL-21 effect on MT-MMP1 is dose-dependent Ve+=protein extracts from the colon of a patient with CD were used as positive control. Panel B of the Figure shows the MT-MMP1/β-actin content ratio in protein extracts prepared from stimulated intestinal myo-fibroblasts as above indicated. Values are expressed in arbitrary densitometric units and indicate mean±standard deviation of 3 separate experiments;

FIG. 12 shows that IL-21 and TNF-α cooperate in enhancing the synthesis of MMPs-1,2,3 e 9 but have no inducing effect on TIMP1 and TIMP2 in intestinal myofibroblasts isolated from a CD patient;

FIG. 13 shows that the addition of CD LPMC supernatants to cultures of CD myofibroblasts enhances MMPs synthesis, and that such an effect may be inhibited by the anti-IL-21 antiserum (GM2) but not control antiserum;

FIG. 14 shows IL-21R expression in intestinal epithelial cells. Panel A shows IL-21R expression, evaluated by immunohistochemistry, in colon specimens from a normal control and a CD patient. Panel B depicts a representative Western blot showing IL-21R and β-actin in total protein extracted from epithelial cells of one normal subject (control), one patient with CD and one patient with UC. Panel C shows the IL-21R/β-actin protein ratio in protein extracts prepared from epithelial cells isolated from the colon of 7 CD patients, 7 UC patients and 7 normal controls. Values are expressed in arbitrary densitometric units (a. u.) and indicate mean±SD of all the experiments. Panel D depicts a representative Western blot showing IL-21 in total protein extracted from 6 different colon cancer epithelial cell lines;

FIG. 15, panel A, shows the effect of IL-21 on the expression of several phosphorylated proteins in DLD-1 cells. A representative Western blot showing phosphorylated proteins (upper blot) and β-actin (lower blot) is shown. Panel B depicts IL-21 effect on protein secretion by DLD-1 cells after 48 hours of culture. The analysis has been carried out using a commercial kit that is able to simultaneously evaluate 120 different proteins. An enhanced MIP-3α secretion by IL-21 stimulated cells (circle marked proteins) was observed. Such an increase was then confirmed by analysis of MIP-3α in supernatants of unstimulated and IL-21-stimulated DLD-1 by ELISA (panel C);

FIG. 16 shows a representative Western blot showing IL-21 (upper blot) and β-actin (lower blot) in total protein extracts prepared from gastric mucosal specimens of 3 patients with Hp infection and 3 normal controls;

FIG. 17 shows a representative Western blot showing IL-21 (upper blot) and β-actin (lower blot) in total protein extracts prepared from duodenal mucosal specimens of 3 patients with celiac disease (celiacs) and 3 normal controls;

FIG. 17 shows a representative Western blot showing IL-21 R in total protein extracted from epithelial cells isolated from the stomach of two patients with Hp-associated gastritis and two normal subjects (controls). Panel B shows a representative Western blot showing IL-21 R and γ-chain in total proteins extracted from gastric cancer epithelial cell lines (AGS and MKN);

FIG. 19, panel A, shows the dose-dependent effect of IL-21 on the secretion of MMP-2 and MMP-9 in epithelial gastric cells (AGS). Panel B shows the ability of IL-21 to enhance NF-kB activation in AGS cells. One of the 3 representative EMSA is shown. Panel C shows how the inhibition of NF-kB activation by TPCK reduces the IL-21-mediated the synthesis of MMP2 and MMP9;

FIG. 20 shows a representative Western blot for IL-21 (upper blot), IL-21R (middle blot) and β-actin (lower blot) in total protein extracts prepared from cutaneous biopsies taken from affected and unaffected areas of 4 patients with psoriasis.

### EXAMPLE 1

### Study of IL-21 Expression in Patients with CD, UC, Gastritis Correlated to Hp Infection, and Celiac Disease

### Materials and Methods

### Patients and Samples

Mucosal samples were taken from freshly obtained specimens of 29 patients with CD. In 18 patients the disease was confined to the terminal ileum, while in the remaining lesions were present in both the ileum and colon. At the time of surgery, 13 patients were receiving steroid therapy, 10 were treated with steroids plus immunouppressors, and 6 were on mesalazine plus antibiotics. 18 patients have a fibrostricturing disease. From patients with ileocolonic involvement, mucosal samples were taken from both involved and spared, ileal and colonic areas.

Additional mucosal samples were taken from the colon of: a) 26 patients with UC undergoing colonoscopy and 4 patients undergoing colectomy for a chronic active course poorly responsive to pharmacological treatment with steroids and immunouppressors; b)6 patients with diverticular disease, 10 patients with irritable bowel syndrome and 26 patients with colon cancer. In the latter case, mucosal samples were taken from macroscopically and microscopically unaffected areas. These samples were considered as normal controls. Additionally, mucosal samples were taken from: 1) stomach of 13 patients with gastritis correlated to Hp infection and 14 patients without macroscopically and microscopically gastritis; b) duodenum of 14 patients with celiac disease and 14 normal controls. Ethical approval was obtained by local committee.

### Intestinallamina Propria Mononuclear Cells (LPMC) Isolation and Culture

LPMC were prepared by the DTT-EDTA and collagenase procedure. An aliquot of LPMC was immediately used for extracting total proteins, while the remaining LPMC were either resuspended in RPMI 1640 (Sigma-Aldrich S.r.l., Milan) supplemented with 10% fetal bovine serum (FBS) (Sigma-Aldrich) and cultured or used to purify T lamina propria lymphocytes (T-LPL). For this purpose, LPMC were incubated for 30 minutes at 4° C. with anti-CD14, CD19 and CD56 antibodies (Miltenyi Biotec S.r.l., Calderara di Reno, Italy) and T-LPL were then collected by negative selection using the magnetic cell sorting system (MACS, Miltenyi Biotec S.r.l.). CD4+, CD8+ and CD45RO+ positive T-LPL were purified with the same methodology using antibodies specific for these cell subtypes (Miltenyi Biotec, S.r.l.).

### Protein Extraction and Western Blotting Analysis

Mucosal samples, LPMC or purified cells were homogenized and total proteins were extracted by using a buffer A containing 10 mM Hepes (pH 7.9), 10 mM KCl, 0.1 mM EDTA and 0.2 mM EGTA. The buffer was added with dithiothreitol 1 mM (DTT), 10 μg/ml aprotinine, 10 μg/ml leupeptine and 1 mM phenylmethansulphonyl fluoride (alle reagents from Sigma-Aldrich).

IL-21 protein was analyzed using a rabbit antibody specific for human IL-21 (0.5 μg/ml, ProSci Incorporated, Poway, Calif., USA).

Horseradish-peroxidase conjugated goat anti-rabbit antibodies (Dako Ltd) were used at final dilution 1:20.000 to detect the primary antibody binding, and immunoreactivity was visualized with a chemiluminescent kit (Pierce, Rockford, Ill., USA). After the analysis of IL-21, blots were stripped and then incubated with an anti-human β-actin antibody (final dilution 1:5000, Sigma-Aldrich) as internal protein loading control.

The intensity of each Western blotting band was analyzed using a computer-assisted scanning densitometry (Total lab, AB.EL Science-Ware Srl, Rome).

### Results

IL-21 was detected in all analyzed samples, but its expression was strongly enhanced in patients with CD (FIG. 1). In addition, an increased expression of IL-21 was seen in patients with UC in comparison to patients with diverticular disease or normal controls (FIG. 1). In patients with CD, the enhanced IL-21 expression was evident in inflamed areas (FIG. 2, upper blot) and was not associated with the type of lesion (FIG. 2, lower blot).

In CD, increased expression of IL-21 was also seen when the analysis was carried out using proteins extracted from LPMC (FIG. 3A). In order to exclude that IL-21 content differences in patients with CD or UC and controls reflected the higher number of mucosal T lymphocytes infiltration, the analysis was also carried out on proteins extracted from the same number of LPL of patients and controls. As shown in FIG. 3, panel B, T-LPL of patients with CD and at a lesser degree of patients with UC contained a higher amount of IL-21. This increase was particularly pronounced in CD4+ cell subtype (left inset of the same figure), and was not dependent on the state of cell activation, as IL-21 content was more pronounced in CD45RO+ LPL isolated from colon of CD patients in comparison to control CD45RO+ LPL (right inset of FIG. 3B).

An increased expression of IL-21 was also demonstrated in the stomach of patients with Hp-associated gastritis (FIG. 16) and in duodenal biopsies of patients with celiac disease (FIG. 17).

### EXAMPLE 2

### Human IL-21 Neutralizing Antibodies Development

### Materials And Methods

### Analysis and Definition of IL-21 Antigenic Sequences

A computerized analysis of the human sequence of IL-21 protein was carried out by Washington Biotechnology Company (Washington, USA) in order to identify potential high antigenicity sites. Therefore, such sequences were considered as optimal epitopes for the immunization and production of antibodies directed against IL-21.

### Peptide Synthesis

Peptide synthesis and purification were carried out by Washington Biotechnology Company. Each peptide was synthesized by the “automated, solid-phase chemistry” technique using Fmoc chemistry. In each peptide sequence, each amino acid was coupled through a peptide bond, and protecting groups were used to avoid unwanted reactions. Excess reagents are washed away at the completion of the synthesis and the complete peptide was then removed from the support resin and purified by a chromatographic technique (reverse phase column chromatography) to give a final product that is better than 90% pure. Each peptide was then analyzed and confirmed by mass spectrometry analysis (MALDI).

### New Zealand White (NZW) Rabbit Immunization

Rabbit NZW immunization was carried out by Washington Biotechnology Company. To induce immunization, each peptide was conjugated to a large protein carrier (KLH) and injected subcutaneously with adjuvant into 2 specific-pathogen-free rabbits (New Zealand White). Following three booster injections over a period of 6 weeks a test bleed to analyse the antiserum by ELISA was taken. A bleed was taken from each rabbit only when the antibody titre was greater than 1:50000 as measured by ELISA. Blood was taken at 6 or 8 weeks following initial immunization. Each antiserum titre was then characterized by ELISA.

### Analysis of Neutralizing Effectiveness of Each Antiserum

To assay the neutralizing activity of each anti-IL-21 antiserum, PBMC were isolated by Ficoll stratification using blood taken from healthy volunteers and resuspended in RPMI containing BSA 0.5% (bovine serum albumin) with or without the initial addition of human recombinant IL-21 (25 ng/ml final concentration) in presence or absence of each anti-IL-21 antiserum or control sera, each used at a final dilution ranging from 1:500 to 1:5000. At the same time experiments using lymphocyte cell lines (Jurkat cells) were carried out as above stated. After 30 minute IL-21 stimulation, cells were collected and used to extract total proteins as above indicated. 200 μg of protein/sample were then separated by electrophoresis, blotted onto nitrocellulose, and analyzed for phosphorylated and total Stat3 content by Western blotting, using monoclonal specific antibodies (Santa Cruz Biotechnology). The intensity of each Western blotting band was then quantified using a computer-assisted scanning densitometry (Total lab, AB.EL Science-Ware Srl).

### Results

As expected stimulation of PBMC with IL-21 was associated with an enhanced Stat3 phosphorylation (FIG. 4). This was evident also when Jurkat cells were used (data not shown). Each anti-IL-21 antiserum was effective to inhibit the IL-21-mediated induction of phosphorylated Stat3 (FIG. 4).

### EXAMPLE 3

### Role of IL-21 in the Modulation of Mucosal Th1 Cell Response

### Materials and Methods

### Cell Culture

To investigate whether IL-21 was involved in Th1 cells differentiation in CD, LPMC isolated as above indicated from inflamed areas of 6 patients with CD, undergoing intestinal resection, were resuspended in RPMI 1640 supplemented with 10% FBS. LPMC were then cultured in the absence or presence of anti-IL-21 antiserum NZW-GM2 (final dilution 1:500) or control serum (1:500) for 12 hours and then stimulated with an anti-CD3 antibody (final dilution 1:500) for further 24 hours.

### Protein Extraction and Western Blotting Analysis

T-bet, p-Stat4 and Stat4 expression was analyzed by Western blotting, as above indicated, using specific antibodies against these proteins (T-bet, and total Stat4 at final dilution 1:500, both manufactured by Santa Cruz Biotechnology, p-Stat4 at 1.5 μg/ml concentration, manufactured by Histo-Line Laboratories, Milan.

The intensity of each Western blotting band was then quantified using a computer-assisted scanning densitometry (Total lab, AB.EL Science-Ware Srl).

### ELISA

LPMC culture supernatants were analyzed for IFN-γ content by a commercially available ELISA kit according the instructions of the manufacturer (Peprotech, London, UK).

### Results

IFN-γ was measurable and p-Stat4 and T-bet expressed in unstimulated LPMC cultures (FIG. 5). LPMC stimulation by anti-CD3 was associated with a marked increase of active Stat4 and T-bet, and this was associated with an enhanced IFN-γ secretion. Importantly, the neutralization of IL-21 activity through the use of GM2 antiserum inhibited the expression of active Stat4 and T-bet induced by anti-CD3, thus resulting in a significant inhibition of IFN-γ (p=0.02) (FIG. 5).

### EXAMPLE 4

### Effect of Anti-IL-21 Antiserum on the Viability of IBD Intestinal I T Lymphocytes

### Materials and Methods

### Patients and Samples

Mucosal samples were taken from surgical specimens of 8 patients with CD, wherein the disease was restricted at the terminal ileum and ascendant colon. At the time surgery, all the patients were receiving steroid and immunosuppressive therapy and have a fibrostricturing disease. Mucosal samples were also taken from the colon of: a) 4 patients with UC undergoing colectomy for a disease poorly responsive to pharmacological treatment with steroids and immunosuppresors; b)6 patients with colon cancer. In this latter case, mucosal samples were taken from macroscopically and microscopically unaffected areas. These samples were regarded as normal controls. Ethical approval was obtained by local committee.

### Intestinal Lamina Propria Mononuclear Cells (LPMC) Isolation and Culture

LPMC were prepared by DTT-EDTA-collagenase procedure. An aliquot of LPMC was immediately used for purifying CD4+ T lymphocytes, as above indicated, while the remaining LPMC were resuspended in RPMI 1640 (Sigma-Aldrich S.r.l., Milan) supplemented with 10% fetal bovine serum (FBS) (Sigma-Aldrich) and cultured.

Both unfractioned LPMC and CD4+ T lymphocytes were cultured in the presence or absence of different dilution of GM2 anti-IL-21 antiserum, or control serum for a time ranging from 2 to 20 hours.

### Cytofluorimetric Analysis of Cell Death and of Mitochondrial Transmembrane Potential

Death cell rate was analyzed by incubating cells for 20 minutes with 5 μg/mL propidium iodide (PI; Sigma-Aldrich) and subsequently stained with a commercial solution of Annexin V (AV) labelled with phycoerythrin (FITC) (Becton Dickinson, Milano). In the same way, AV percentage was evaluated in CD3+ T lymphocytes by identifying the latter through the use of a human monoclonal antibody anti-CD3 (Becton Dickinson). Cell fluorescence was then measured using FL-1 and FL-2 channels of a cytofluorimeter FACSCalibur (Becton Dickinson). For the analysis of the mitochondrial transmembrane potential, cells were incubated with 20 μM 3,3′-dihexyloxacarbocyanine iodide (DiOC6(3) (Invitrogen S.r.L., Milano) during the last 20 minutes of culture, and then the fluorescence was evaluated by cytofluorimetry.

### Results

After 20 hours culture, the addition of anti-IL-21 antiserum (GM2) to the LPMC culture dramatically reduced CD3+ lymphocytes survival. Cytofluorimetric analysis shows that anti-IL-21 significantly enhanced the percentage of AV positive cells (28,5±18), PI (7±1,4) and AV/PI (41±7,6) in comparison to unstimulated cells (AV: 11±1; PI: 1,5±0, 16; AV/PI:20,8±5,7) or treated with control serum (AV: 11,6±0,69; PI: 2±0,7; AV-PI:21±5,77) (P<0.01) (FIG. 6).

The anti-IL-21 enhanced the percentage of positive AV and PI CD4+ T lymphocytes (data not shown), thus excluding the possibility that the anti-IL-21 effect occurs through a mechanism of antibody-mediated cytotoxicity. These anti-IL-21 effects were associated with a marked loss of mitochondrial transmembrane potential (FIG. 7).

### EXAMPLE 5

### IL-21 Receptor Expression in Myofibroblasts and Mucosal Epithelial Cells and Role of IL-21 in the Induction of MMPS and MIP-3α

### Materials and Methods

### Isolation of Intestinal Myo-Fibroblasts

LPMC, from both CD (N=8) and UC(N=8) patients and normal controls (N=8), isolated as above indicated, were resuspended in MEM 1×, containing 1% not essential amino acids, and 10% FBS, and incubated for 24 hours at 37° C. Not adherent cells were then removed, and plates adherent cells were incubated in the presence of the aforementioned culture medium. After 3 passages, the resulting myo-fibroblast population was morphologically and phenotypically characterized, using specific monoclonal antibodies.

### Isolation of Gastric and Intestinal Epithelial Cells

Gastric and intestinal epithelial cells were isolated from endoscopic biopsies taken from the antrum of 8 patients with gastritis by Hp and 8 patients without gastritis (normal controls), and from the colon of 8 patients with CD, 8 patients with UC and 8 normal controls. Biopsy samples were immediately washed in Hank's solution, then incubated for 30 minutes in a solution containing 1 mM EDTA. Finally, the resulting cell population was extensively purified by Percoll gradient stratification (Sigma-Aldrich).

### Intestinal Myo-Fibroblasts Cultures and Fetal Intestinal Fibroblast Lines

Myo-fibroblasts of patients with CD, UC and normal control subjects, as well as the fetal intestinal fibroblast lines, CCD18CO, were maintained in MEM 1×, containing 1% not essential amino acids, and 10% FBS, until the confluence was reached. Then, cells were maintained in MEM1X in absence of FBS for 24 hours, and finally stimulated or not with different doses of IL-21 for 48 hours. In the same way, myo-fibroblasts were stimulated with 50 ng/ml of IL-21 in presence of 25 ng/ml of TNF-α. To examine whether IL-21 produced by CD LPMC was able to modulate MMP synthesis, LPMC isolated from the intestine of 3 patients with CD were placed in culture as above indicated, and after 48 hours, supernatants were collected and frozen at −80° C. until use. Such supernatants were then added to intestinal myofibroblasts cultures of patients with CD at dilution 1:20, with or without the initial addition of anti-IL-21 antiserum (GM2), at final dilution of 1:500, or of control serum. After 48 hours of culture, myofibroblasts supernatants were collected and analyzed for MMP content.

To examine whether IL-21R expression can be modulated, myo-fibroblasts isolated from the intestine of normal subjects were stimulated with TNF-α (20 ng/ml) or IL-1β(20 ng/ml) for 24 hours, and IL-21R content was evaluated by Western blotting.

### AGS Cultures

AGS were maintained in DMEM/F12 containing 10% FBS, until the confluence was reached. Then, cells were maintained in DMEM/F12 in absence of FBS for 24 hours, and finally stimulated or not with different doses of IL-21 for a time ranging from 5 minutes to 48 hours. In parallel, AGS cultures were treated with TPCK, an inhibitor of NF-kB activity (1-10 μM) for 60 minutes, before being stimulated with IL-21 (50 ng/ml) for additional 48 hours.

### EMSA

Stimulated and unstimulated AGS as above indicated were homogenized and cytoplasmic extracts were collected using buffer A containing 10 mM Hepes (pH 7.9), 10 mM KCl, 0.1 mM EDTA, and 0.2 mM EGTA. Nuclear extracts were prepared by solubilization of the remaining nuclei in buffer C containing 20 mM Hepes (pH 7.9), 0.4 M NaCl, 1 mM EDTA, 1 mM EGTA, and 10% glycerol. Both buffers were supplemented with 1 mM dithiothreitol (DTT), 10 μg/ml aprotinin, 10 μg/ml leupeptin, and 1 mM phenylmethansulphonyl fluoride (Sigma). DNA binding studies of nuclear proteins were carried out for 20 minutes at room temperature in a reaction volume of 20 μl containing 10 mM Tris-HCl, 50 mM KCl, 1 mM DTT, 2.5% of glycerol, 5 mM MgCl₂, 1 μg Poly (dl-dC), (Sigma) 50 fmol of probes containing biotin labelled oligonucleotides and 5 μg of nuclear proteins.

DNA probes were prepared using two consensus oligonucleotides (FWD, 5′:-AGTTGAGGGGAGTTTCCCAGG-3′ (SEQ ID NO:7), REV, 5′:-CGGACCCTTTCAGGGGAGTTGA-3′ (SEQ ID NO:8)), that were biotin 3′ labelled using a commercial kit (Pierce, Rockford, Ill., U.S.A.). The binding specificity was verified by incubating nuclear proteins samples with unlabeled NF-kB probes or with unlabeled oligonucleotides of the IL gene (IL2G), (5′:ACAACGCGTGAGCTCTCTAGAAAGCATCAT CTCAACACTAACTTGATAATTAAGTGCCTCGAGCACA-3′ (SEQ ID NO:9)) in molar excess 100 times greater to saturate the binding.

In neutralization experiments, a human monoclonal antibody anti NF-kB/p65 (Santa Cruz Biotechnology) or a control antibody (Dako Ltd) (both used at 2.5 μg/20 μl concentration) were incubated with nuclear proteins 45 minutes before adding the probes. A not denaturing 6% polyacrylamide gel was employed for electrophoretic separation. After blotting onto membrane, labelled oligonucleotides were detected by EMSA chemiluminescent kit (Pierce).

DLD-1 culture

DLD-1 were maintained in RPMI1640 containing 10% FBS, until the confluence was reached. Then cells were maintained in RPMI1640 in absence of FBS for 24 hours, and finally stimulated or not with different IL-21 doses for 15 minutes to 48 hours.

### IL-21R Immunohystochemistry

IL-21R expression was evaluated in intestinal resection specimens of patients with CD and normal controls. Tissue sections were cut, deparafinized and dehydrated following xilene and ethanol treatment and then the slides were incubated in microwave oven for 20 minutes in citrate buffer (0.01 M), pH 6 (Sigma-Aldrich). The incubation with human monoclonal antibodies anti-IL-21R(R&D Systems) or control antibodies used at dilution 1:20, was carried out at 4° C. overnight. The staining specificity was confirmed using blocking peptides. After TBS washings (Sigma), slides were incubated with a HRP peroxidase conjugated secondary antibody (dilution 1:50, Dako SpA, Milan) for 30 minutes at room temperature.

Immunoreactive cells were seen by adding diaminobenzidine (Sigma) as substrate and contrasting with hematoxylin. Control sections were prepared under the same immunohistochemical conditions, as previously described, by replacing the primary antibody with a control antibody (Dako). After being dehydrated following xilene and ethanol treatment slides were analyzed by optical microscope.

### IL-21R Western Blotting

Western blot analysis for IL-21R was performed using total proteins extracted. Extracts were prepared from gut myo-fibroblasts cell line, CCD18CO, from gastric and intestinal epithelial cells, as well as gastric (AGS) and intestinal cancer epithelial cells (HT-29, HT-115, Colo205, DLD-1, T84, Caco-2). Protein extraction and IL-21R analysis were carried out as above indicated. To examine IL-21R a commercial monoclonal antibody (final concentration 1 μg/ml, R&D Systems) was used.

### MMPs Western Blotting

Following incubation, supernatants of myo-fibroblasts and AGS cultures, either left unstimulated or stimulated with IL-21, were collected, centrifuged and used for the analysis of MMP-1, MMP-2, MMP-3, MMP-9, TIMP-1, and TIMP-2 by Western blotting, using specific monoclonal antibodies (R&D Systems). Additionally, protein extracts were prepared from IL-21 stimulated and unstimulated cells and then used for the analysis of MT-MMP-1 by Western blotting, using a specific monoclonal antibody (R&D Systems).

### Protein Array System

To analyze whether IL-21 stimulation of intestinal epithelial cells was associated with changes of the content of proteins/cytokines/chemokines, DLD-1 culture supernatants, either unstimulated or stimulated with IL-21, were subjected to a protein analysis by using a commercial Protein Array System kit, that is able to analyze 120 proteins simultaneously (RayBiotech, Inc. Norcross, Ga., USA). 1 ml of each supernatant was incubated overnight onto a membrane containing the antibodies against 120 proteins. After several washings, the blot was incubated with a biotin labelled secondary antibody, followed by the incubation with streptavidin and substrate. The intensity of each band was evaluated by computer-assisted analysis.

### MIP-3α ELISA

MIP-3α was analyzed in the DLD-1 culture supernatants, either unstimulated or stimulated with IL-21 as above indicated, by a commercial ELISA kit (R&D Systems, Space Import-Export Srl, Milan). Optical density was measured by the ELISA reader Dynatech MR 5000 at the wavelength of 450 nm. Results were expressed in pg/ml.

### Results

IL-21R was found to be constitutively expressed by intestinal primary myo-fibroblasts and fetal intestinal fibroblasts (CCD18CO) (FIG. 8). The same cell types also expressed the common γ-chain receptor, an essential component of the functional IL-21R (FIG. 8). IL-21R expression was further enhanced by inflammatory stimuli, including TNF-α and IL-1β (FIG. 9).

Importantly, stimulation of intestinal myofibroblasts with IL-21 enhanced MMPs secretion, but not TIMPs. This was evident both in primary myo-fibroblasts and CCD18CO (FIG. 10). IL-21 also enhanced the expression of membrane MMP such as MT-MMP-1 (FIG. 11) whose activity is essential for the activation of MMP-2. Additionally IL-21 cooperated with TNF-α in stimulating MMPs synthesis (FIG. 12). To confirm the biological role of IL-21 in the induction of MMPs, it was demonstrated that CD LPMC supernatants promoted MMPs synthesis by intestinal myofibroblasts and that such an effect was inhibited by adding the anti-IL-21 antiserum GM2 (FIG. 13). A constitutive expression of IL-21R was also seen in intestinal primary epithelial cells and in colon cancer epithelial lines (FIG. 14). Importantly, immunoistochemistry (FIG. 14, panel A) and Western blotting (FIG. 14, panel B) analysis showed an enhanced expression of IL-21R in epithelial cells of patients with IBD, particularly with CD. To examine the effect of IL-21 on biological activity of intestinal epithelial cells, the DLD-1 cell line was selected as these cells express IL-21R. In vitro stimulation of these cells with IL-21 was associated with changes in the content of different phosphorylated proteins (FIG. 15, panel A). Cell response to IL-21 was supported by the evidence that DLD-1 secreted high levels of MIP-3a in response to IL-21 stimulation (FIG. 14, panels B and C). IL-21R expression was not restricted to the intestinal epithelium, since gastric primary epithelial cells and gastric cancer epithelial cell lines such as AGS and MKN constitutively expressed the receptor (FIG. 18). Consistently, stimulation of AGS with IL-21 resulted in enhanced secretion of MMP-2 and MMP-9 (FIG. 19, panel A), two gelatinases produced in excess in the epithelium of patients with Hp infection. This effect seems to be dependent on IL-21 ability to regulate the activation of NF-kB, given that IL-21 increased NF-kB activation (FIG. 19, panel B), and inhibition of NF-kB by TPCK reduced the IL-21-mediated MMP-2 and MMP-9 synthesis (FIG. 19, panel C).

### EXAMPLE 6

### Study of the Expression of IL-21 and IL-21R in Patients with Psoriasis

### Materials and Methods

### Patients and Samples

Cutaneous biopsy samples were taken from affected and unaffected areas of 9 patients with psoriasis. At the time of recruiting, no patient was receiving therapy. Biopsies were immediately frozen in liquid nitrogen and maintained at −80° C. until use.

### IL-21 and IL-21R Western Blotting

Total protein extracts from each sample were analysed for IL-21 and its receptor using the above mentioned methodologies.

### BIBLIOGRAPHY

Nickoloff B J, et al. J Clin Invest 2004; 113:1664-75.
Firestein G S. J Clin Invest 2004; 114:471-4.
Sollid L M. Nat Rev Immunol 2002; 2:647-55.
Podolsky D. K., N. Engl. J. Med., 2002; 347:417-29.
Campbell I K, et al. Immunol Cell Biol. 2003; 81:354-66.
Spahn J D, et al. J. Asthma. 1997; 34:177-94.
Scholmerich J. Aliment Pharmacol Ther. 2004; 20 Suppl 4:66-74.
Seegers D, et al. Aliment Pharmacol Ther, 2002; 16:53-58.
Sandborn J, et al. Gastroenterology, 2002; 122:1592-608.
Fiocchi C. J Clin Invest, 2001; 108:523-26.
Habib T, et al. J Allergy Clin Immunol 2003; 112:1033-45.
Parrish-Novak J, et al. J Leukoc Biol. 2002; 72:856-63.
Collins M, et al. Immunol Res. 2003; 28:131-40.
Zhang J L, et al. Biochem Biophys Res Commun. 2003; 300:291-6.
Mehta D S, et al. J. Immunol. 2003; 170:4111-8.
Jin H, et al. J Immunol 2004; 173:657-65.
Ma H L, et al. J. Immunol. 2003; 17:608-15.
Wurster A L, et al. J Exp Med. 2002; 196:969-77.
Gallegos A M, et al. Cell 2004; 117:149-51.
Neurath M F, et al. J Exp Med 1996; 183:2605-2616.
Monteleone G, Gastroenterology. 1997; 112:1169-78.
Monteleone G, Gastroenterology. 1999; 117:1069-77.
Heuschkel R B, et al. Gut 2000; 47:57-62.
Saarialho-Kere U K, et al. Am J Pathol 1996; 148:519-26.
Kane D, et al. J Rheumatol 2004; 31:1274-80.
Wall S J, et al. Br J. Dermatol. 2003; 149:13-6.
Kwon J H, et al. Gut. 2002; 51:818-26.
Dieu-Nosjean M C, et al. J Exp Med 2000; 192:705-18.
Mori N, et al. Gastroenterology 2003; 124:983-92.
EP 1443 055.

1. Use of the antibodies specific and selective for at least one of the following antigenic epitopes:

| | |
|---|---|
| KMI HQH LSS RTH GSE DS; | (SEQ ID NO: 1) |
| NVS IKK LKR KPP STN; | (SEQ ID NO: 2) |
| LGT LVH KSS SQG QDR; | (SEQ ID NO: 3) |
| TNA GRR QKH RLT CPS; | (SEQ ID NO: 4) |
| CDS YEK KPP KEF LER; | (SEQ ID NO: 5) |
| CFQ KAQ LKS ANT GNN E; | (SEQ ID NO: 6) |

for the preparation of a medicament for the treatment of Th1-mediated immune-inflammatory diseases associated with an altered IL-21 expression.

2. Use of the antibodies specific and selective for at least one of the following antigenic epitopes:

| | |
|---|---|
| KMI HQH LSS RTH GSE DS; | (SEQ ID NO: 1) |
| NVS IKK LKR KPP STN; | (SEQ ID NO: 2) |
| LGT LVH KSS SQG QDR; | (SEQ ID NO: 3) |
| TNA GRR QKH RLT CPS; | (SEQ ID NO: 4) |
| CDS YEK KPP KEF LER; | (SEQ ID NO: 5) |
| CFQ KAQ LKS ANT GNN E; | (SEQ ID NO: 6) |

for the in-vitro diagnosis of Th1-mediated immune-inflammatory disease associated with an altered IL-21 expression.

3. Use according to claim 1, wherein the Th1-mediated immune-inflammatory diseases associated with an altered IL-21 expression are chronic inflammatory bowel diseases, celiac disease, psoriasis, or Hp associated gastritis.

4. Use according to claim 3, wherein said chronic inflammatory bowel diseases, are Crohn's disease or ulcerative colitis.

5. Use according to claim 1, wherein said antibodies are labeled with a fluorescent dye, a radioisotope or a drug.

6. Use according to claim 1, wherein said antibodies are polyclonal or monoclonal, antibody fragments, chimeric or single chain antibodies.

7. Diagnostic kit comprising at least one of the antibodies specific and selective for at least one of the following antigenic epitopes and/or the same epitopes:

| | |
|---|---|
| KMI HQH LSS RTH GSE DS; | (SEQ ID NO: 1) |
| NVS IKK LKR KPP STN; | (SEQ ID NO: 2) |
| LGT LVH KSS SQG QDR; | (SEQ ID NO: 3) |
| TNA GRR QKH RLT CPS; | (SEQ ID NO: 4) |
| CDS YEK KPP KEF LER; | (SEQ ID NO: 5) |
| CFQ KAQ LKS ANT GNN E. | (SEQ ID NO: 6) |

8. An isolated antibody that is specifically reactive with at least one of:
amino acids 146-162 of human IL-21 (SEQ ID NO: 1);
amino acids 97-111 of human IL-21 (SEQ ID NO: 2);
amino acids 20-34 of human IL-21 (SEQ ID NO: 3);
amino acids 110-124 of human IL-21 (SEQ ID NO: 4);
amino acids 125-139 of human IL-21 (SEQ ID NO: 5); or
amino acids 78-93 of human IL-21 (SEQ ID NO: 6).

9. The antibody of claim 8, wherein the antibody is an antibody fragment.

10. A method for reducing or inhibiting an IL-21 mediated inflammatory response in a cell, the method comprising exposing the cell to an effective amount of an antibody of claim 8 to reduce or inhibit the inflammatory response in the cell.

11. A method for reducing or inhibiting an IL-21 mediated inflammatory response in a subject in need thereof, the method comprising administering an effective amount of an antibody of claim 8.

12. A method of treating an immune-inflammatory disease associated with altered IL-21 expression in a subject in need thereof, the method comprising administering an effective amount of an antibody of claim 1.

13. The method of claim 12, wherein the immune-inflammatory disease associated with altered IL-21 expression is selected from the group consisting of chronic inflammatory bowel diseases (IBD), celiac disease, and psoriasis.

14. The method of claim 13, wherein the chronic inflammatory bowel disease is Crohn's disease.

15. The method of claim 13, wherein the chronic inflammatory bowel disease is ulcerative colitis.

16. (canceled)

17. A method of treating psoriasis in a subject in need thereof, comprising administering an effective amount of an antibody of claim 8.
